(19) United States
(12) Patent Application Publication
(10) Pub. No.: US 2011/0112363 A1
(43) Pub. Date: May 12, 2011

(54) ENDOSCOPE

(75) Inventors: Takehiko Koga, Kanagawa (JP); Issei Suzuki, Kanagawa (JP)

(73) Assignee: FUJIFILM CORPORATION, Tokyo (JP)

(21) Appl. No.: 12/942,791

(22) Filed: Nov. 9, 2010

(30) Foreign Application Priority Data
Nov. 11, 2009 (JP) 2009-258098
Mar. 24, 2010 (JP) 2010-068961

(51) Int. Cl. A61B 1/04

(52) U.S. Cl. 600/109

(57) ABSTRACT

On a head section connected to a distal end of an inserter of an endoscope, an air/water nozzle, an observation window and a ring projection are provided. On the ring projection, a first inclined plane and a vertical plane are formed. The first inclined plane inclines such that the outer diameter thereof gradually decreases from the top surface of the observation window toward a flat surface. The vertical plane is positioned at the counter side from the air/water nozzle across the observation window. Fluid injected from the air/water nozzle hits the first inclined plane and spreads to pass over the observation window. The vertical plane stops backflow of a water drop on the flat surface toward the observation window, so as to prevent that the water drop reaches on the top surface of the observation window.

### BACKGROUND OF THE INVENTION

1. Field of the Invention

The present invention is related to an endoscope which comprises a fluid injection nozzle which injects fluid to an observation window.

2. Description of the Related Arts

An endoscope comprises an observation window for taking image light of a subject, and a fluid (air/water) injection nozzle which injects fluid to the observation window, at a head of an inserter for the subject. The observation window and the fluid injection nozzle are provided on a flat surface which is approximately perpendicular to the axial direction of the inserter. The observation window is provided such that a surface as a light entrance surface is protruded a certain height from the flat surface. On the top surface of the observation window, secretion and dirt from the subject are adhered. To wash them, the fluid injection nozzle injects water from an injection orifice toward the observation window. Then air is injected from the injection orifice so as to blow water drops remained on the top surface of the observation window. Since remaining dirt and water drops on a part of the observation window hinder observation of the subject, it is preferable that fluid injected from the fluid injection nozzle spreads all over the top surface of the observation window.

For this purpose, in endoscopes disclosed in Japanese Laid-open Patent Publication No. 2003-210388 and Japanese Laid-open Patent Publication No. 03-165731, an inclined plane whose height gradually becomes higher from the flat surface to the top surface of the observation window is formed around all over the periphery of the observation window. According to the inclined plane, a step between the top surface of the observation window and the flat surface is eliminated so that fluid injected from the fluid injection nozzle flows smoothly toward the top surface of the observation window and spreads all over the top surface of the observation window. The fluid passed through the top surface of the observation window runs down a slope of the inclined plane at the other side of the fluid injection nozzle, and reaches the flat surface. As described above, the inclined plane improves a washing function for the observation window and a draining function for the injected water.

However, in the endoscopes of Japanese Laid-open Patent Publication No. 2003-210388 and Japanese Laid-open Patent Publication No. 03-165731, since the inclined plane whose height gradually becomes higher from the flat surface to the top surface of the observation window is formed around all over the periphery of the observation window, there is a problem that water injected from the fluid injection nozzle and once passed the top surface of the observation window comes back to the top surface of the observation window through the inclined plane. The backflow of water clouds a sight for observation. In this condition, the observation window needs to be washed again. Therefore, a frequency of washing has to be increased.

### SUMMARY OF THE INVENTION

An object of the present invention is to provide an endos cope in which a washing function for an observation window and a draining function for injected water are improved without increasing a frequency of washing.

In order to achieve the above and other objects, an endoscope of the present invention comprises a flat surface which is provided at a distal end of an inserter to be inserted into a subject and is approximately perpendicular to the axial direction of the inserter, an observation window which is positioned on the flat surface and has a surface as a light entrance surface being protruded a certain height from the flat surface, a fluid injection nozzle which is positioned on the flat surface and injects fluid toward the observation window, a first inclined plane which is disposed around at least a part of the periphery of the observation window facing the fluid injection nozzle and inclines such that whose height gradually decreases from the periphery of the observation window toward outside, and a back-flow prevention member which is provided on the counter side from the fluid injection nozzle across the observation window so as to prevent that fluid injected from the fluid injection nozzle and passed the surface of the observation window flows backwardly from the flat surface toward the surface of the observation window.

It is preferable that the back-flow prevention member is a plane which is positioned at the periphery of the observation window and stands from the flat surface in a height direction in which the surface of the observation window is projected, the plane being a vertical plane standing vertically from the flat surface or being a second inclined plane having an acute angle with the flat surface. The vertical plane includes a plane having an approximately right angle (85° to 95°) with the flat surface . In addition, the second inclined plane is not limited to be flat, and may be curved.

It is preferable that the first inclined plane has a width covering an injection area of fluid from the fluid injection nozzle.

It is preferable that the back-flow prevention member is disposed along the periphery of the observation window.

It is preferable that the back-flow prevention member is disposed along a rest portion of the periphery of the observation window where the first inclined plane is not disposed.

It is preferable that a link surface which smoothly links between the first inclined plane and the second inclined plane is formed at a boundary between the first inclined plane and the back-flow prevention member, so that there is no edge portion at the boundary.

It is preferable that each of the first inclined plane and the back-flow prevention member covers approximately each half of the periphery of the observation window.

It is preferable that the back-flow prevention member covers between the observation window and an illumination window disposed near the observation window.

It is preferable that the back-flow prevention member side of the flat surface with reference to the boundary between the back-flow prevention member and the first inclined plane has a height lower than that of the first inclined plane side of the flat surface.

It is preferable that the back-flow prevention member is integrally formed with a member on which the flat surface is formed.

It is preferable that the back-flow prevention member is formed on another member which is different from a member on which the flat surface is formed. The other member may be for example a lens barrel to which the observation window is attached, the peripheral surface of the lens barrel working as the back-flow prevention member. In addition, the other member may be the observation window itself, the flange of the observation window working as the back-flow prevention member.

It is preferable that the inserter comprises a suction opening which is provided on the flat surface and sucks liquid around it, and a third inclined plane which is provided around the observation window and inclines toward the suction opening, with passing through between a projection projected from the flat surface and the observation window, and around the back-flow prevention member.

It is preferable that the inserter comprises a connecting member which is provided around the observation window and connects between a projection projected from the flat surface and the observation window.

It is preferable that the connecting member covers an area between the observation window and the projection, and wherein the back-flow prevention member is a cut-out formed by cutting a part of the connecting member positioned at the counter side from the fluid injection nozzle across the observation window. In addition, it is preferable that the flat surface is formed such that the cut-out side thereof has a height lower than that of the first inclined plane side thereof.

According to the present invention, since there are the inclined plane which is disposed the position facing the fluid injection nozzle, and the back-flow prevention member which is provided on the counter side from the fluid injection nozzle across the observation window so as to prevent that fluid injected from the fluid injection nozzle and passed the surface of the observation window flows backwardly from the flat surface toward the surface of the observation window, the washing function for the observation window and the draining function for the injected water can be improved without increasing a frequency of washing.

### BRIEF DESCRIPTION OF THE DRAWINGS

The above and other subjects and advantages of the present invention will become apparent from the following detailed description of the preferred embodiments when read in association with the accompanying drawings, which are given by way of illustration only and thus are not limiting the present invention. In the drawings, like reference numerals designate like or corresponding parts throughout the several views, and wherein:

FIG. 1 is a perspective view of the exterior of an endoscope system;

FIG. 2 is a perspective view illustrating a configuration of a head section of an electronic endoscope;

FIG. 3 is a cross-sectional view along A-a line of FIG. 2;

FIG. 4A is an explanatory drawing illustrating a state that fluid is injected toward an observation window;

FIG. 4B is an explanatory drawing illustrating a state that a water drop adheres on a flat surface;

FIG. 5 is a perspective view illustrating an example of a second embodiment in which a second inclined plane making an acute angle with the flat surface is formed as a back-flow prevention member;

FIG. 6 is a cross-sectional view along B-b line of FIG. 5;

FIG . 7A is a perspective view illustrating a boundary area between the inclined plane and the back-flow prevention member of FIG. 5;

FIG. 7B is a perspective view illustrating an example in which a link surface to smoothly link the inclined plane with the back-flow prevention member is formed on the boundary area;

FIG. 8 is a perspective view illustrating an example of a third embodiment in which a peripheral surface of a lens barrel exposed from a cut-out functions as the back-flow prevention member;

FIG. 9 is a plan view of the example of FIG. 8;

FIG. 10 is a perspective view illustrating a variation of the third embodiment;

FIG. 11 is a plan view of the example of FIG. 10;

FIG. 12 is a plan view illustrating an example in which a first inclined plane is provided in a fluid injection range, and a rest portion of the periphery of the observation window works as the back-flow prevention member;

FIG. 13 is a cross-sectional view illustrating an example of a fourth embodiment in which a projection and a protective cover are provided separately, and the projection is fixed to the flat surface;

FIG. 14 is an example in which the projection and the protective cover are provided separately, and the projection is formed integrally with a cylindrical member which is fitted with the protective cover;

FIG. 15 is an example in which the projection and the protective cover are provided separately, and the projection is formed integrally with the lens barrel which holds the observation window and is fitted with the protective cover;

FIG. 16 is a perspective view illustrating an example of a fifth embodiment in which a third inclined plane is provided around the observation window;

FIG. 17 is a plan view of the example of FIG. 16;

FIG. 18 is a perspective view illustrating an example of a sixth embodiment in which a connecting member is provided between the observation window and an illumination window;

FIG. 19 is a perspective view illustrating an example in which a connecting member covering all area between the observation window and the illumination window is provided;

FIG. 20 is a plan view of the example of FIG. 19; and

FIG. 21 is a perspective view illustrating an example in which a recessed edge along a cut-out formed on the connecting member is provided on the flat surface.

### DETAILED DESCRIPTION OF THE PREFERRED EMBODIMENTS

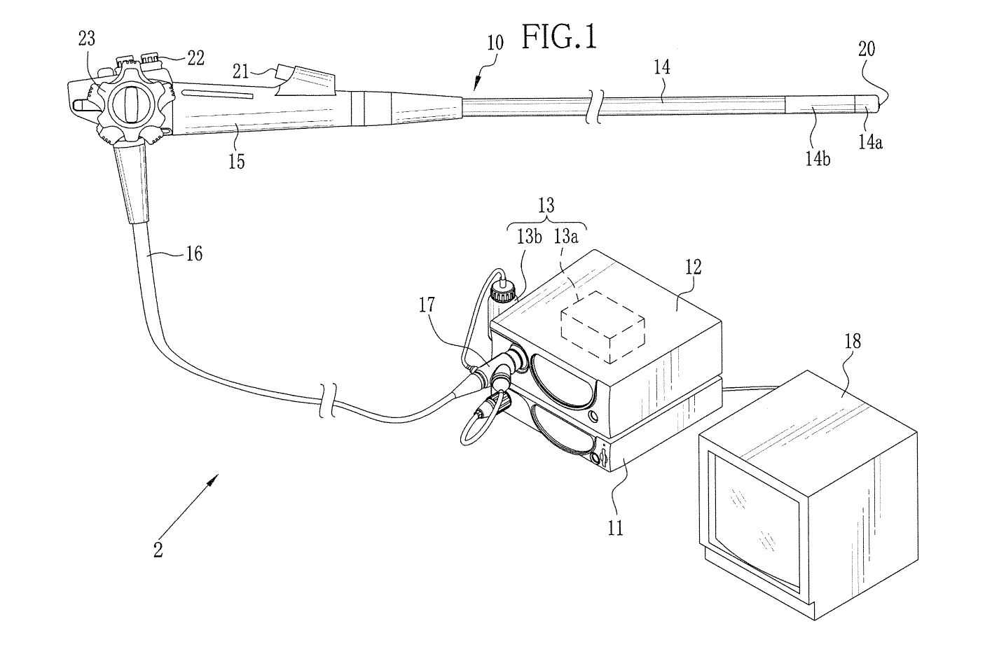

As illustrated in FIG. 1, an endoscope system 2 comprises an electronic endoscope 10, a processor unit 11, a light source unit 12, an air/water injection unit 13 and so on. The air/water injection unit 13 includes a known air injector (pump or the like) 13a which is integrated in the light source unit 12 to inject air, and a water tank 13b provided outside the light source unit 12 to store wash water. The electronic endoscope 10 includes a flexible inserter 14 to be inserted into a subject, a control handle 15 connected to the proximal end of the inserter 14, and a universal cord 16 connected to the processor unit 11 and the light source unit 12.

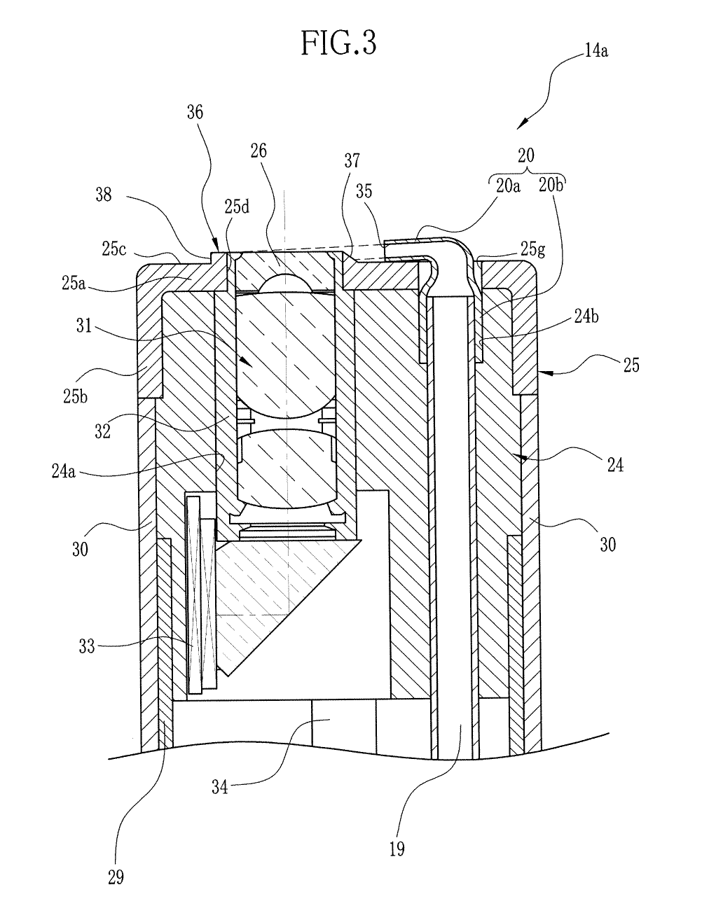

To the distal end of the inserter 14, a head section 14a, in which a CCD 33 (see FIG. 3) as an image pickup device for the subject and so on are integrated, is connected. At the back of the head section 14a, a bending section 14b constituted of a plurality of joint pieces is provided.

At the distal end of the universal cord 16, a connector 17 is attached. The connector 17 is a composite connector to which the processor unit 11, the light source unit 12 and the air/water injection unit 13 are connected.

The processor unit 11 applies various image processing to an image signal input from the CCD 33 through the universal cord 16 and the connector 17 so as to convert the image signal into a video signal, and sends a drive control signal for controlling operations of the CCD 33. The video signal converted in the processor unit 11 is displayed as an endoscopic image on a monitor 18 which is connected to the processor unit 11 through a cable. In addition, the processor unit 11 is electrically connected to the light source unit 12 and controls all operations of the endoscope system 2.

Inside the inserter 14 and the control handle 15, an air/water channel 19 (see FIG. 3) is provided. The air/water channel 19 is connected to an air/water nozzle (fluid injection nozzle) 20 provided in the head section 14a. In addition, the air/water channel 19 passes through the universal cord 16 and is connected to the air/water injection unit 13.

In the control handle 15, an forceps opening 21 through which various operation tools such as those having an injection needle or a high-frequency knife at their distal ends is inserted, an air/water button 22, an angle knob 23 and so on are provided. When the air/water button 22 is operated to perform the air injection, air from the air injector 13a is sent to the air/water nozzle 20. When the air/water button 22 is operated to perform the water injection, wash water is sent to the air/water nozzle 20 from the water tank 13b by pressure of air from the air injector 13a. The air/water nozzle 20 selectively injects the air and the wash water fed through the air/water channel 19.

In addition, when the angle knob 23 is operated, a wire extending through the inserter 14 is pushed and pulled to bend the bending section 14b in the up, down, right and left directions . Thus, the head section 14a can be directed toward a desired direction inside a body cavity (the subject).

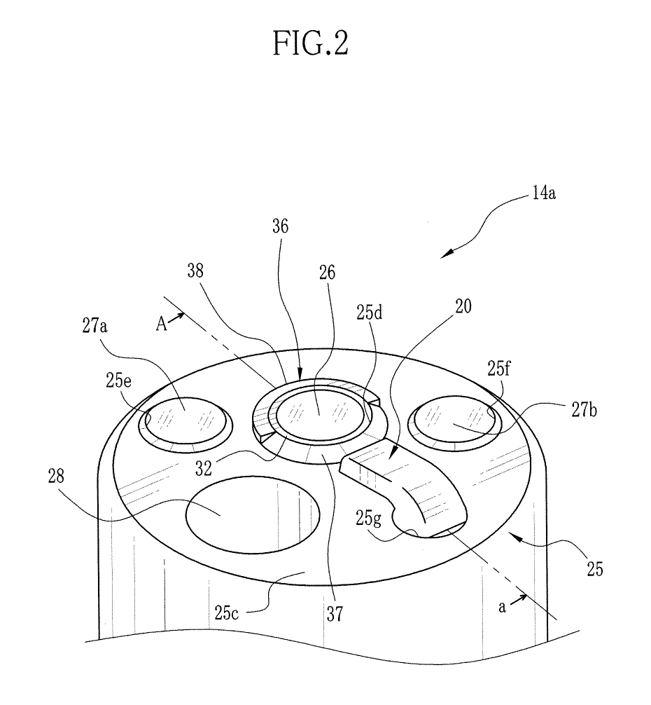

As illustrated in FIG. 2 to FIG. 4, the head section 14a includes a head section main body 24, a cap-shaped protective cover 25 attached to the distal end side of the head section main body 24, an observation window 26, illumination windows 27a and 27b, a forceps outlet 28 and the air/water nozzle 20. The proximal end of the head section main body 24 is connected to a joint piece 29 at the distal end of the bending section 14b.

The protective cover 25 has a head plate section 25a which covers the distal end side of the head section main body 24, and a cylindrical member 25b which covers the peripheral surface of the head section main body 24. An external tube 30 which covers the peripheral surface of the bending section 14b extends to the head section main body 24, and the distal end of the external tube 30 and the proximal end of the cylindrical member 25b are faced and fixed to each other with use of an adhesive or the like. On the head plate section 25a, a flat surface 25c which is approximately perpendicular to the axial direction of the inserter 14 and configures the distal end face of the inserter 14 is formed. On the head plate section 25a, through holes 25d to 25g through which the observation window 26, the illumination windows 27a and 27b, and the air/water nozzle 20 are exposed from the flat surface 25c, and the forceps outlet 28 are formed.

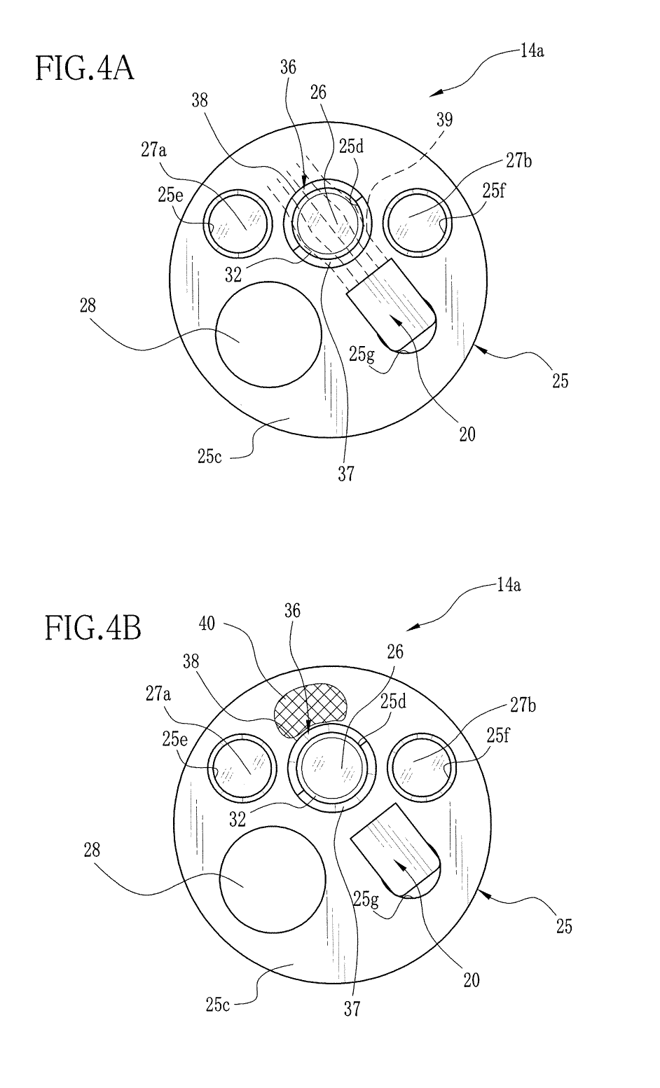

Note that the vertical direction of FIG. 4A and FIG. 4B corresponds to the up-down direction for bending the bending section 14b. The observation window 26 is positioned on the upper side of the flat surface 25c, and the air/water nozzle 20 and the forceps outlet 28 are positioned below the observation window 26. The illumination windows 27a and 27b are positioned symmetrically against the observation window 26.

The observation window 26 is an objective lens which is the distal end lens of an objective lens unit 31, and also is a cover glass. The observation window 26 has a circular shape. A lens barrel 32, which holds the optical system of the objective lens unit 31 and covers a peripheral surface (a flange) of the observation window 26, is formed such that the distal end surface of the lens barrel 32 is on the same level with the surface (the light entrance surface) of the observation window 26. The lens barrel 32 fits into through holes 24a, 25d of the head section main body 24 and the protective cover 25, and is attached such that the top surface of the observation window 26 protrudes a predetermined height from the flat surface 25c. The predetermined protrusion height is for example 0.3 mm.

Note that as the observation window 26, a cover glass which is positioned at the distal end of the objective lens unit 31 and has no lens effect may be used. In addition, the observation window 26 is not required to constitute the objective lens unit 31. For example, the observation window 26 as the cover glass may be directly fitted and fixed into the through hole 25d of the protective cover 25.

Behind the objective lens unit 31, the CCD 33 is attached. The CCD 33 is for example an interline transfer CCD. However, the image pickup device is not limited to the CCD 33, and a CMOS may be used as the image pickup device.

The illumination windows 27a and 27b work also as illumination lenses which illuminate illumination light from the light source unit 12 to an observed region in the subject. Each of the illumination windows 27a and 27b faces the exit end of a light guide (not illustrated). The light guide is formed by bundling a plurality of optical fibers (for example made of quartz). The light guide passes through the inserter 14, the control handle 15, the universal cord 16 and the connector 17, for guiding the illumination light from the light source unit 12 to the illumination windows 27a and 27b. The forceps outlet 28 is connected to a forceps channel (not illustrated) which is connected to the forceps opening 21 of the control handle 15. When a medical instrument is inserted into the forceps opening 21, its distal end is exposed from the forceps outlet 28.

The air/water nozzle 20 has an injection tube section 20a at the distal end side, and a connecting tube section 20b at the proximal end side which are integrally formed. The connecting tube section 20b fits around the distal end side peripheral surface of the air/water channel 19, to make a connection with the air/water channel 19. The connecting tube section 20b and the air/water channel 19 fit into a through hole 24b of the head section main body 24. The injection tube section 20a is formed in a tubular shape that smoothly curves approximately 90° from the connecting tube section 20b to an injection orifice 35 at the distal end, and is exposed outside through the through hole 25g of the protective cover 25. As a flow of fluid from the injection tube section 20a is illustrated by dashed lines in FIG. 3, the injection tube section 20a is formed such that fluid is sprayed from a position diagonally above the head section 14a toward the observation window 26.

On the protective cover 25, a ring projection 36 which projects a certain height from the flat surface 25c is integrally formed between the periphery of the observation window 26 and the flat surface 25c. The ring projection 36 has an inner peripheral surface continuous with the through hole 25d, and has a first inclined plane 37 and a vertical plane 38 which works as a back-flow prevention member. Note that since water drops are remained on the top surface of the observation window 26 inside the ring projection 36 when the ring projection 36 is projected from the top surface of the observation window 26, it is preferable that the protrusion height of the ring projection 36 is equal to or slightly less than the height of the top surface of the observation window 26.

The first inclined plane 37 is disposed along the periphery of the observation window 26 to follow the shape of the observation window 26, more concretely, to follow the shape of the periphery of the lens barrel 32 which is positioned around the outer periphery of the observation window 26. The first inclined plane 37 is inclined such that whose height is gradually reduced from the periphery of the observation window 26 toward outside, concretely, such that whose height is reduced from the height of the top surface of the observation window 26 to the height of the flat surface 25c. The first inclined plane 37 is provided at a position facing the injection orifice of the air/water nozzle 20. The first inclined plane 37 covers about halfway around the periphery of the observation window 26, including a width of the fluid injection range where the air/water nozzle 20 injects fluid. Note that in this embodiment, the lens barrel 32 is disposed between the flange of the observation window 26 and the flat surface 25c. However, the observation window 26 may be directly fitted to the protective cover 25.

Unlike the first inclined plane 37, the vertical plane 38 is a plane which extends vertically in the height direction in which the top surface of the observation window 26 protrudes from the flat surface 25c. Concretely, the vertical plane 38 stands up with an approximately right angle of 85° to 95° against the flat surface 25c. The vertical plane 38 is positioned at the counter side of the air/water nozzle 20 across the observation window 26, and is disposed along the rest portion of the periphery of the observation window 26 where the first inclined plane 37 is not disposed. That is, the first inclined plane 37 and the vertical plane 38 covers all around the periphery of the observation window 26, the inclined plane 37 covers around the air/water nozzle 20 side, and the vertical plane 38 covers around the counter side (the rest) of the periphery of the observation window 26.

Processes for washing the observation window 26 by injecting fluid from the air/water nozzle 20 will be explained with reference to FIGS. 4A and 4B. As described above, the observation window 26 and the first inclined plane 37 are positioned in the fluid injection range of the air/water nozzle 20. As illustrated in FIG. 4A, a part of fluid (air or wash water) 39 injected from the air/water nozzle 20 directly contacts the observation window 26, and the rest of the fluid 39 strikes against the first inclined plane 37, spreads around the peripheral direction of the observation window 26 and ascends the first inclined plane 37. Accordingly, the fluid spreads the whole surface of the observation window 26 to blow off liquid and dirt adhered on the observation window 26. In addition, the wash water is blown off by the air injection.

Then the water including the liquid and the dirt and being passed the observation window 26 falls toward the counter side from the air/water nozzle 20. That is, the water passes the vertical plane 38 and falls toward the flat surface 25c. As illustrated in FIG. 4B, a part of the water passed the observation window 26 becomes a water drop (dirty water after the washing) 40, and remains on the flat surface 25c at the counter side from the air/water nozzle 20.

In this state, if for example the bending section 14b is bent or the inserter 14 is rotated in the axial direction, the water drop 40 may flow toward the top surface of the observation window 26. However, the vertical plane 38 blocks the water drop 40 to prevent that the backflow of the water drop 40 reaches the top surface of the observation window 26. Accordingly, it is prevented that the water drop 40 once blown off is adhered on the observation window 26.

As described above, although there may be the case that the water drop (dirty water after the washing) 40 passed the observation window 26 lefts on the counter side from the air/water nozzle 20, the vertical plane 38 prevents the backflow of the water drop toward the observation window 26. Accordingly, there is no need to blow off the water drop 40 again by injection of wash water or air. Therefore, the washing function for the observation window 26 and the draining function for the injected water are improved without increasing a frequency of washing.

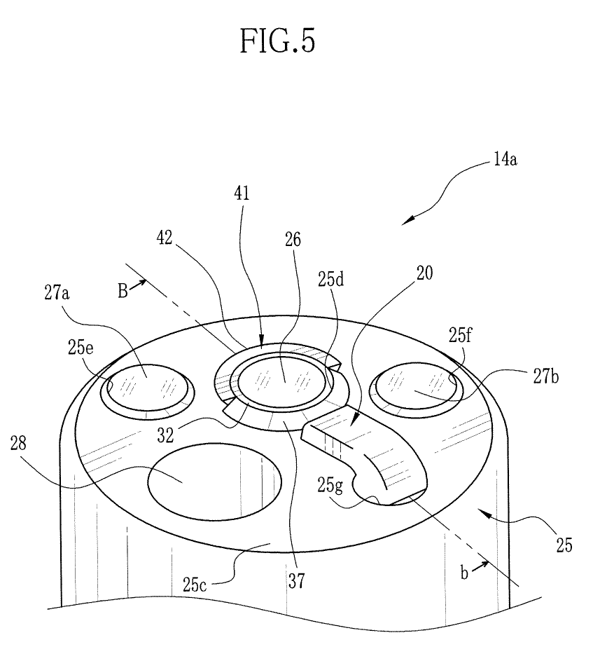

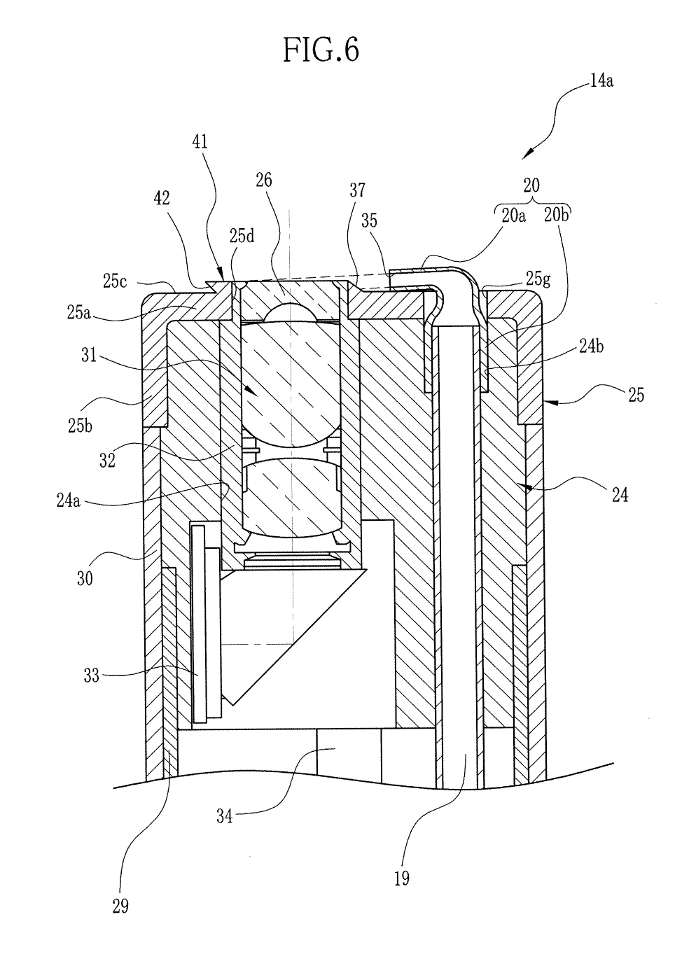

In the first embodiment, the vertical plane 38 as the back-flow prevention member is formed on the ring projection 36. However, the back-flow prevention member is not limited to the vertical plane. As a second embodiment illustrated in FIG. 5 and FIG. 6, a second inclined plane 42 which is inclined from the top end of a ring projection 41 to the flat surface 25 may be used. As same as the vertical plane 38, the second inclined plane 42 is the face which extends from the flat surface 25c in the height direction in which the top surface of the observation window 26 protrudes. However, as different from the vertical plane 38, an angle between the second inclined plane 42 and the flat surface 25c is an acute angle. This configuration more certainly prevents that the water drop 40 reaches the top surface of the observation window 26, compared to the first embodiment. Accordingly, the backflow is more effectively prevented. Note that as the second inclined plane 42, both a flat surface and a curved surface may be used.

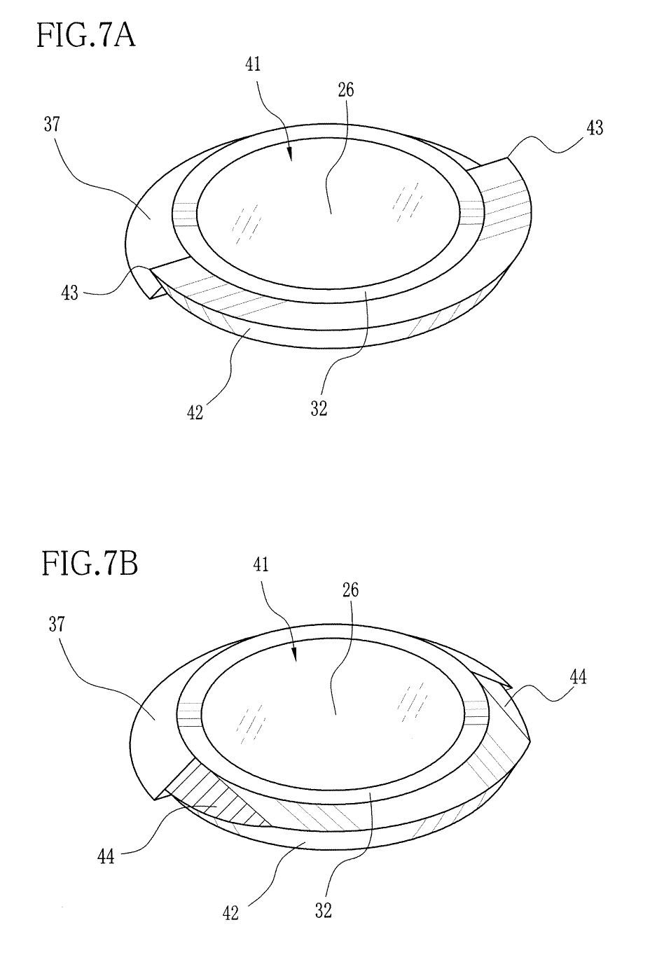

As illustrated in FIG. 7A, since the first inclined plane 37 and the second inclined plane 42 have the inclination directions opposite to each other, there becomes an edge portion 43 at the boundary between the first inclined plane 37 and the second inclined plane 42. In this configuration, when fluid is injected from the air/water nozzle 20 toward the observation window 26 and the first inclined plane 37, the edge portion 43 becomes an obstacle for flow of the fluid, by shielding the flow of the fluid or changing the flow direction of the fluid. To avoid this problem, as illustrated in FIG. 7B, the edge portion 43 illustrated in FIG. 7A may be planed off to form a link surface 44 which smoothly links between the first inclined plane 37 and the second inclined plane 42.

Note that also in the first embodiment in which the vertical plane 38 is formed as the back-flow prevention member, there becomes an edge portion (see FIG. 2) at the boundary between the first inclined plane 37 and the vertical plane 38 . The edge portion may be planed off to form a link surface which smoothly links between the first inclined plane 37 and the vertical plane 38.

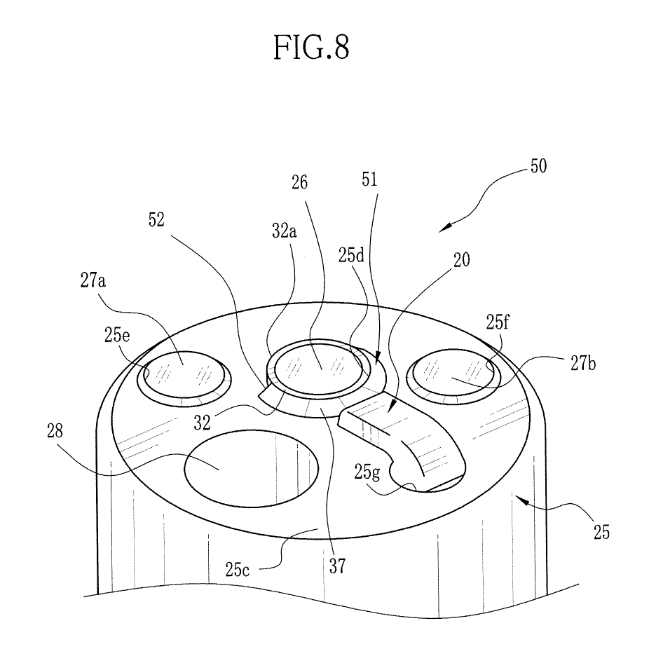

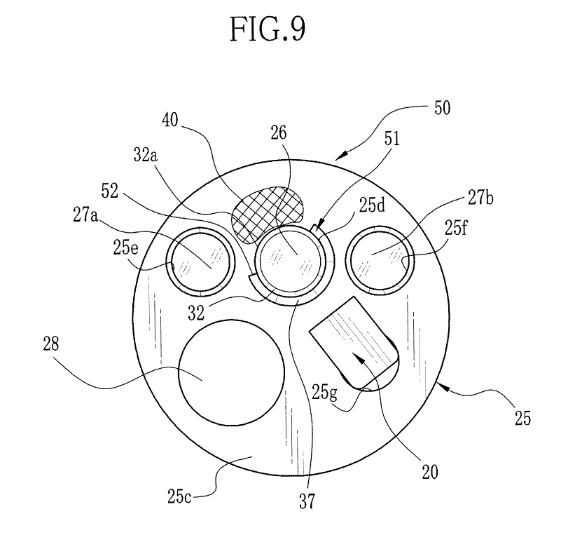

In the first and second embodiments, the first inclined plane 37 and the back-flow prevention member are integrally formed with the ring projection. However, as a head section 50 of a third embodiment illustrated in FIGS. 8 and 9, a circular arc projection 51 having the first inclined plane 37 may be provided, and the peripheral surface 32a of the lens barrel 32 holding the observation window 26 may be exposed from a cut-out 52 formed on the circular arc projection 51, as the back-flow prevention member.

In the head section 50 of the third embodiment, between the periphery of the observation window 26 and the flat surface 25c, the circular arc projection 51 which protrudes the same height as the observation window 26 from the flat surface 25c is integrally provided with the protective cover 25. The circular arc projection 51 has an inner peripheral surface which links the through hole 25d, and has the first inclined plane 37 formed in the fluid injection range of the air/water nozzle 20. The first inclined plane 37 is provided at the position facing the air/water nozzle 20 and covers approximately half of the periphery of the observation window 26, as same as in the first and second embodiments. On the other half of the periphery of the observation window 26 , the cut-out 52 is formed at the counter side of the air/water nozzle 20 across the observation window 26. The cut-out 52 exposes a part of the peripheral surface 32a of the lens barrel 32 which covers the flange of the observation window 26. The part of the peripheral surface 32a exposed from the cut-out 52 works as the back-flow prevention member.

As the third embodiment in which the back-flow prevention member is constituted of the peripheral surface 32a of the lens barrel 32, also in case that the back-flow prevention member is constituted of a different member than the protective cover 25 in which the flat surface 25c is formed, the backflow of the water drop 40 can be prevented as same as the first embodiment.

Note that in this embodiment, the peripheral surface 32a is the vertical plane. However, the peripheral surface 32a may have the inclination same as of the second inclined plane 42 of the second embodiment. In addition, instead of the peripheral surface 32a, the flange of the observation window 26 may become the back-flow prevention member.

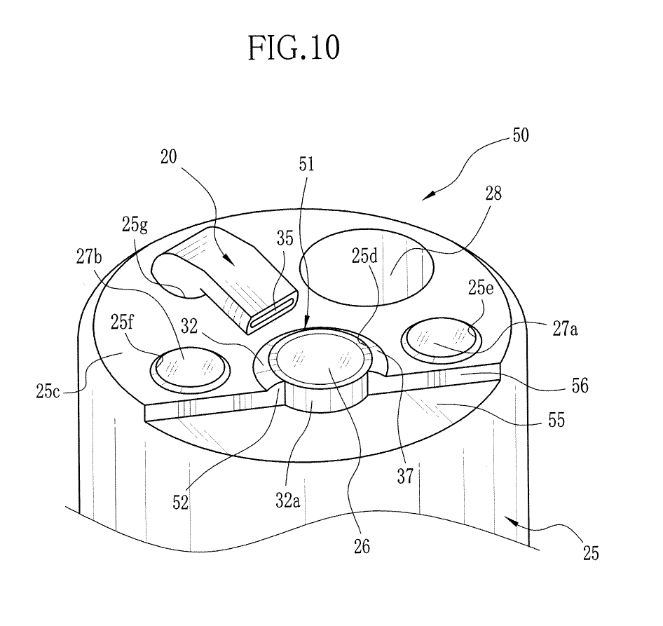

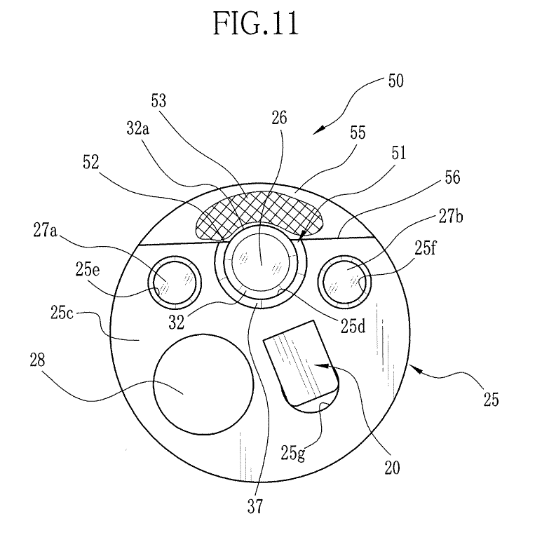

As a variation of the third embodiment illustrated in FIGS. 10 and 11, on the flat surface 25c, a recessed edge 55 may be formed such that the back-flow prevention member side of the flat surface 25c with reference to the boundary between the back-flow prevention member and the first inclined plane 37 has a height lower than that of the first inclined plane side of the flat surface 25c. The recessed edge 55 covers an area from a step 56 as the boundary with the rest of the flat surface 25c to the outer periphery of the protective cover 25. According to this configuration, as illustrated in FIG. 11, a water drop 53 remains on the recessed edge 55 which has a large difference in level with the top surface of the observation window 26, it is more effectively prevented that the water drop 53 flows onto the top surface of the observation window 26, compared to the second embodiment.

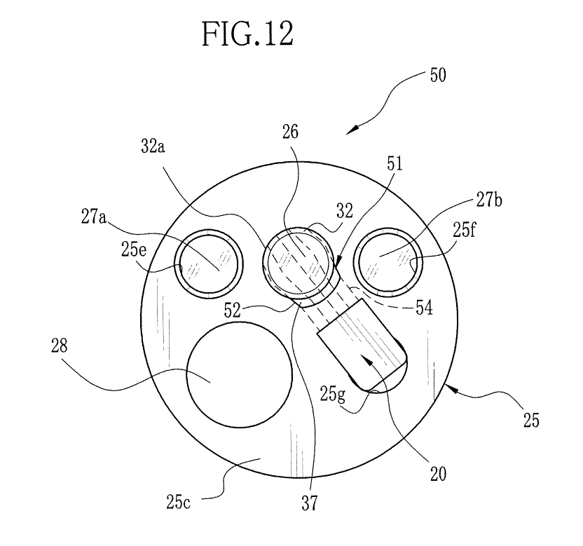

In the above embodiments, the first inclined plane 37 covers approximately half of the periphery of the observation window 26, the covered length is not limited to above. For example, as illustrated in FIG. 12, the first inclined plane 37 may cover only inside a fluid injection range 54 of the air/water nozzle 20, and the rest of the periphery of the observation window 26 may become the back-flow prevention member.

Around the observation window 26, there are projections such as the illumination windows 27a and 27b which are projected from the flat surface 25c, as same as the observation window 26. It is preferable that the first inclined plane 37 is not disposed near the illumination windows 27a and 27b, and the back-flow prevention member is disposed between the observation window 26 and the illumination windows 27a and 27b. According to this configuration, even when the fluid injected from the air/water nozzle 20 hits the illumination windows 27a and 27b near the observation window 26 and the water drop bounces, the back-flow prevention member stops the flow of the water drop to prevent the adhesion of the water drop on the observation window 26.

In the above embodiments, the ring projection 36 or the circular arc projection 51 on which the back-flow prevention member is formed is integrally formed with the protective cover 25. However, the back-flow prevention member and the protective cover 25 may be formed as separate members.

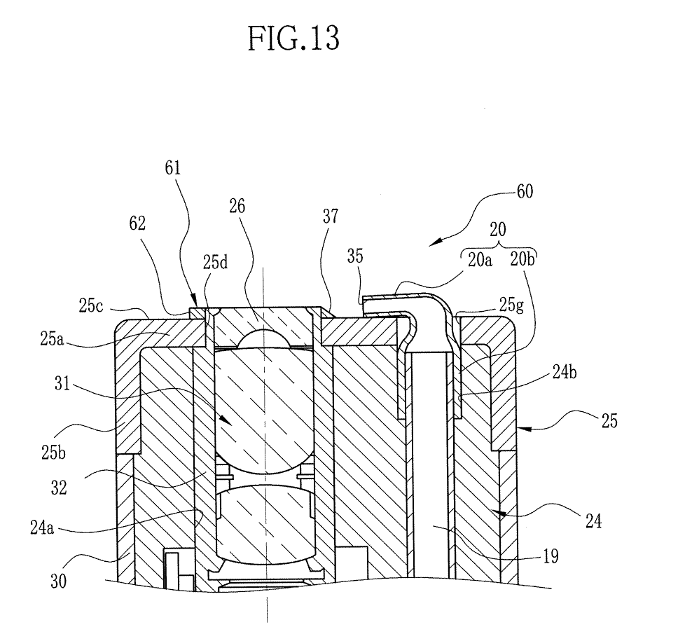

As a fourth embodiment illustrated in FIG. 13, on a head section 60, there are the first inclined plane 37 and a back-flow prevention member 62. The back-flow prevention member 62 is formed on a projection 61 having a shape of a ring or a circular arc which is provided separately from the protective cover 25. The projection 61 is fixed on the flat surface 25c with use of an adhesive or the like. As the above-described ring projection 41, in case the second inclined plane 42, whose outer diameter gradually decreases from the periphery of the observation window 26 to the flat surface 25c, is formed integrally with the protective cover 25 and is used as the back-flow prevention member, since the inclination directions of the first and second inclined planes 37 and 42 are opposite to each other, a construction of a forming die for them becomes complicated, which causes an increase in a cost. However, in the embodiment of FIG. 13 in which the back-flow prevention member is the member different than the protective cover 25, the forming process becomes simple and the cost increase can be prevented.

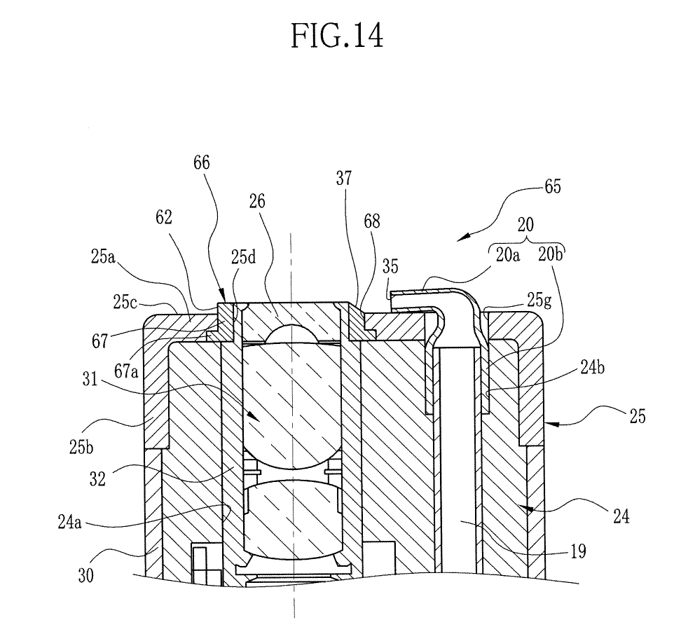

In addition, as a head section 65 illustrated in FIG. 14, a projection 66 having a shape of a ring or an circular arc which has the first inclined plane 37 and the back-flow prevention member 62 and is provided separately from the protective cover 25 may be formed integrally with a cylindrical member 67 which fits around the peripheral surface of the lens barrel 32. The cylindrical member 67 has a flange 67a having a larger outer diameter at the proximal end side (the side facing the head section main body) thereof. Since the flange 67a is engaged with the head plate section 25a, detachment of the cylindrical member 67 is prevented.

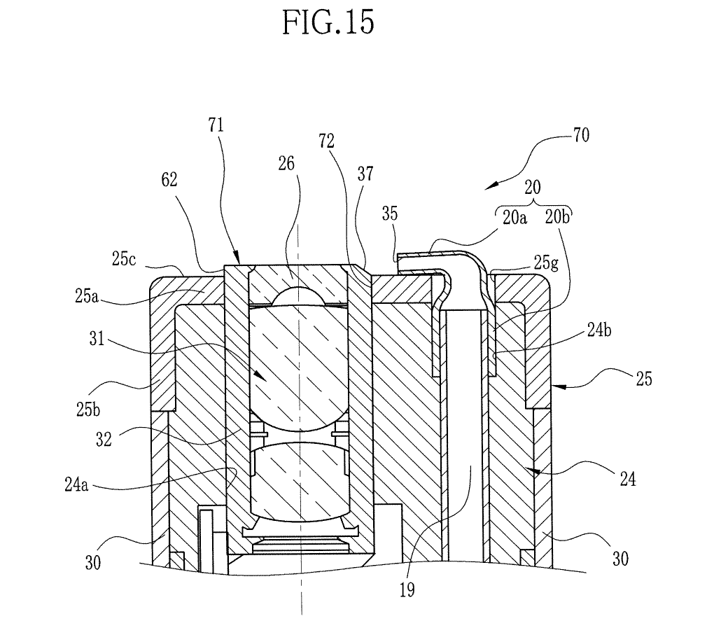

In addition, as a head section 70 illustrated in FIG. 15, a projection 71 having a shape of a ring or an circular arc which has the first inclined plane 37 and the back-flow prevention member 62 and is provided separately from the protective cover 25 may be formed integrally with the lens barrel 32 . The projection 71 is positioned at the distal end of the lens barrel 32 and can be attached such that the peripheral surface of the lens barrel 32 is fitted into a fitting hole 72 formed on the protective cover 25 with the projection 71 protruding a certain height from the flat surface 25c of the protective cover 25.

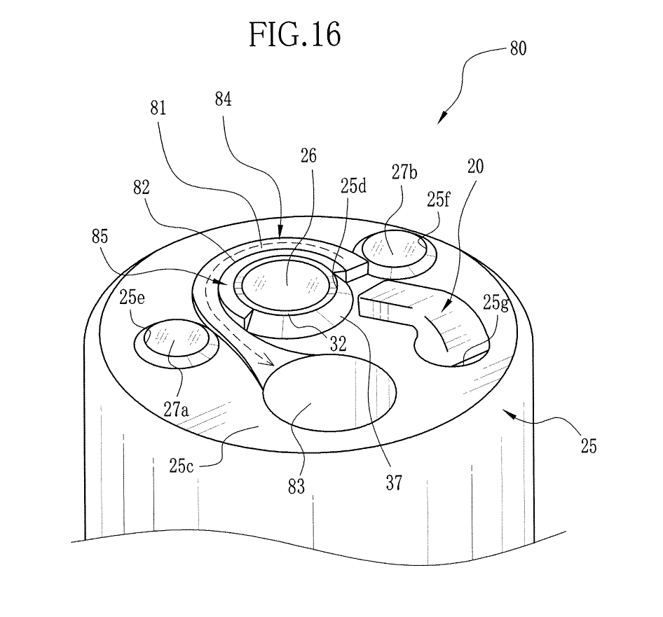

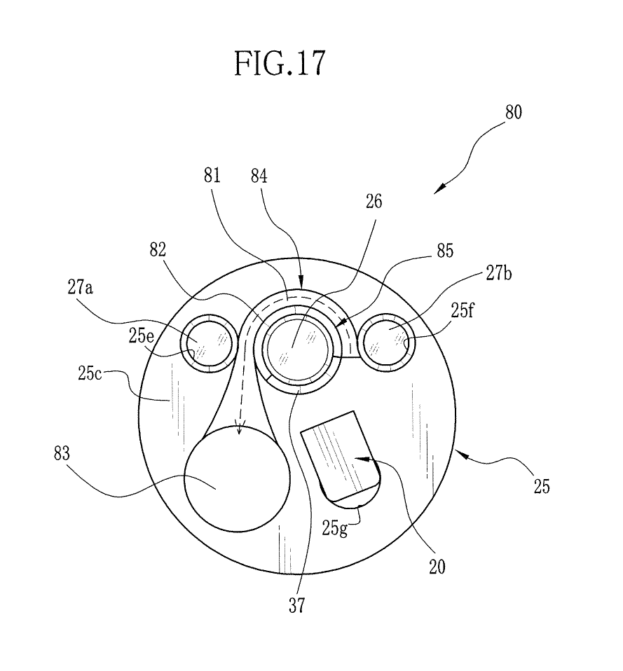

A head section 80 of a fifth embodiment illustrated in FIGS. 16 and 17 provides a third inclined plane 81 for removing a water drop left between the illumination windows 27a and 27b and the observation window 26 which are the projections projected from the flat surface 25c. The head section 80 further provides a curved projection 84 which curves from around the observation window 26 and a back-flow prevention member 82 toward a forceps outlet 83, and a projection 85 which has the first inclined plane 37 and the back-flow prevention member 82. The third inclined plane 81 is formed on the curved projection 84. In addition, the shape of the back-flow prevention member 82 is the same as that of the back-flow prevention members in the first to fourth embodiments.

The curved projection 84, as same as the projections 36, 41, 51, 61, 66, and 71 in the first to fourth embodiments, may be provided integrally with or separately from the protective cover 25. For example, the curved projection 84 may be provided integrally with the lens barrel 32. The head section 80 comprises the forceps outlet 83. The forceps outlet 83 works also as a suction opening, and the forceps channel communicated to the forceps outlet 83 works also as a suction channel. The forceps/suction channel is connected to a suction pump (not illustrated) to suck liquid near the forceps outlet 83.

The third inclined plane 81 inclines from a position contacting the periphery of the back-flow prevention member 82 between the observation window 26 and the illumination window 27b toward the forceps outlet 83, with passing through around the periphery of the back-flow prevention member 82 and between the observation window 26 and the illumination window 27a (an arrow of dashed line indicates the inclination direction).

Processes for washing the observation window 26 by injecting fluid from the air/water nozzle 20 will be explained. The fluid injected from the air/water nozzle 20 directly contacts the observation window 26, and strikes against the first inclined plane 37 to spread the whole surface of the observation window 26. Accordingly, liquid and dirt adhered on the observation window 26 is blown off.

Then the water including the liquid and the dirt and being passed the observation window 26 falls toward the counter side from the air/water nozzle 20. That is, the water passes the back-flow prevention member 82 and falls toward the third inclined plane 81 and the flat surface 25c. A part of the water flows into the third inclined plane 81 and the flat surface 25c becomes a water drop (dirty water after the washing) , and remains on the third inclined plane 81. Especially the water drops, remained between the observation window 26 and the illumination windows 27a and 27b, diffusely reflect illumination light and possibly disturbs a field of view. When the diffuse reflection is caused, an operator activates the suction pump connected to the forceps channel. By suction force from the forceps channel, the water drop flows along the third inclined plane 81 and is sucked into the forceps outlet 83. As described above, by the third inclined plane 81, the water drop remained between the observation window 26 and the illumination windows 27a and 27b can be easily removed. Note that about the water drop remained on the flat surface 25c at the counter side from the air/water nozzle 20, the back-flow prevention member 82 prevents the backflow of the water drop toward the observation window 26.

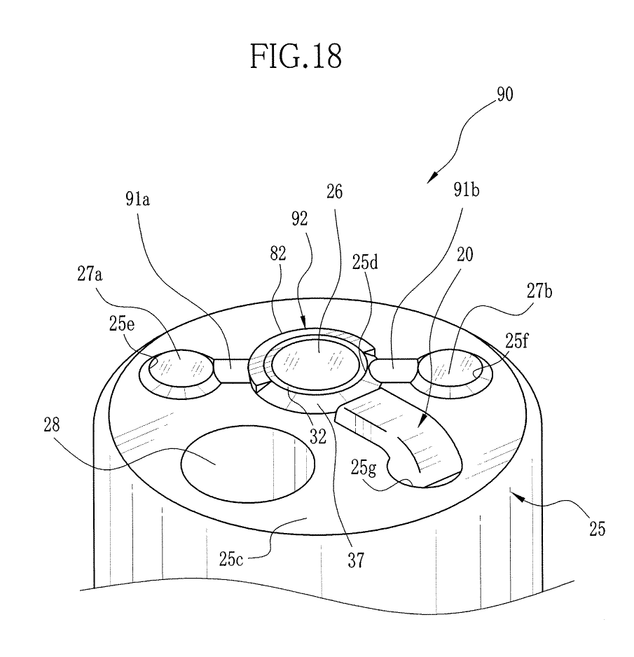

A head section 90 of a sixth embodiment illustrated in FIG. 18 comprises connecting members 91a and 91b which connect between the observation window 26 and the illumination windows 27a and 27b which are the projections projected from the flat surface 25c. On the head section 90, a projection 92 having the first inclined plane 37 and the back-flow prevention member 82 is provided. Accordingly, in addition to the effects of the first to fourth embodiments, it is prevented that a water drop and dirt are remained between the illumination windows 27a and 27b and the observation window 26.

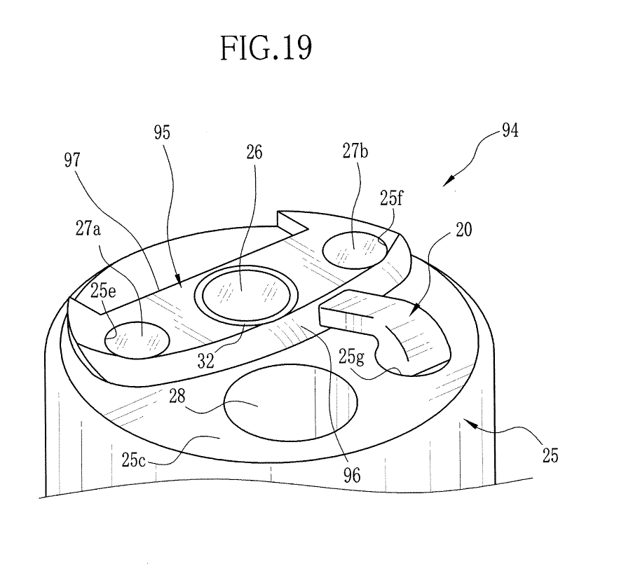

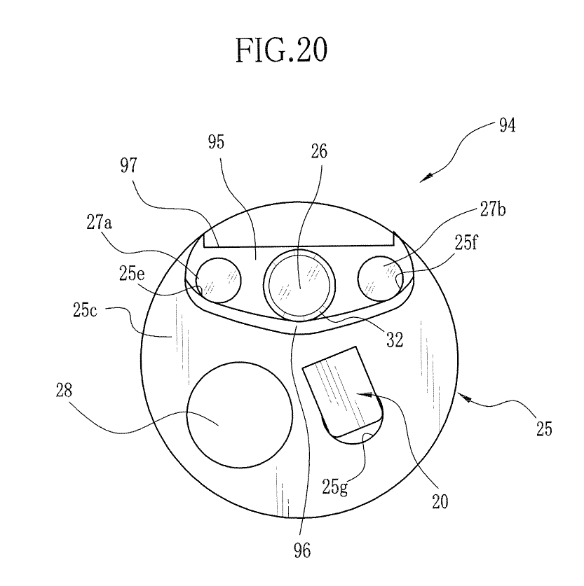

As a head section 94 illustrated in FIGS. 19 and 20 which is a variation of the sixth embodiment, a connecting member 95 which covers areas between the illumination windows 27a and 27b and the observation window 26 may be formed. The connecting member 95 is formed in a shape of an approximately straight line, since the illumination windows 27a and 27b are symmetrically arranged across the observation window 26. A first inclined plane 96, which is provided at a position facing with the injection orifice of the air/water nozzle 20 and has a height gradually decreasing from the periphery of the observation window 26 toward outside, is formed on the connecting member 95. The first inclined plane 96 continues from the periphery of the observation window 26 to the peripheries of the illumination windows 27a and 27b, through the periphery of the connecting member 95.

On the connecting member 95, there is a cut-out 97 formed by cutting a part of the connecting member 95 positioned at the counter side from the air/water nozzle 20 across the observation window 26. Each surface of the cut-out 97 stands from the flat surface in the height direction in which the top surface of the observation window 26 protrudes. In this example, the cut-out 97 works as the back-flow prevention member in which a water drop remains . Accordingly, it is prevented that the water drop flows onto the surface of observation window 26. Note that in FIGS. 19 and 20, the cut-out 97 has an approximately U-shape. However, the shape of the cut-out 97 is not limited to this, and the connecting member 95 may be cut into a shape of a straight line or a curved line.

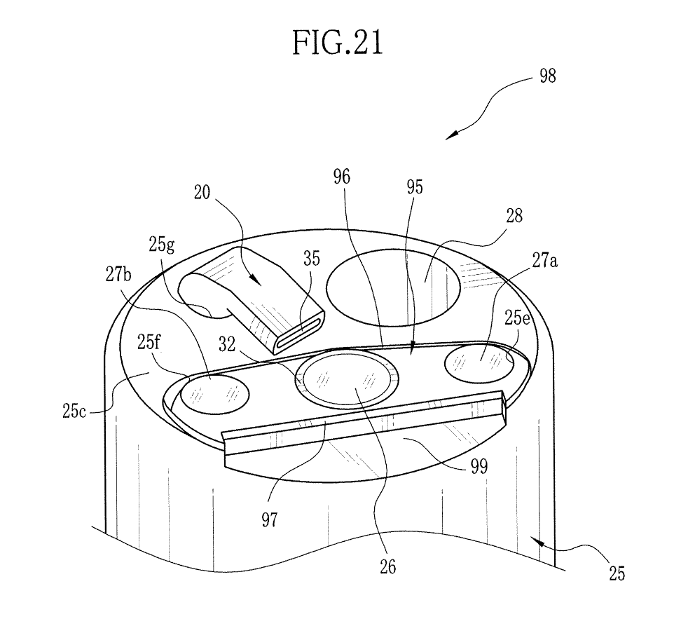

As a head section 98 illustrated in FIG. 21 which is another variation of the sixth embodiment, a recessed edge 99 may be formed on the flat surface 25c such that the cut-out 97 side of the flat surface 25c has a height lower than that of the first inclined plane 96 side of the flat surface 25c, in addition to the above-described connecting member 95 having the cut-out 97. The recessed edge 99 continues from the cut-out 97 to the outer periphery of the protective cover 25. Accordingly, the water drop remains on the recessed edge 99 which has a large difference in level with the top surface of the observation window 26, it is prevented that the water drop flows onto the top surface of the observation window 26.

In the above embodiments, the electronic endoscope which uses an imaging device to make an image representing a state of a subject is explained. However, the present invention is not limited to this, and may be applied to an endoscope using an optical image guide to observe a state of a subject.

Although the present invention has been fully described by the way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An endoscope comprising:
a flat surface which is provided at a distal end of an inserter to be inserted into a subject and is approximately perpendicular to the axial direction of said inserter;
an observation window which is positioned on said flat surface and has a surface as a light entrance surface being protruded a certain height from said flat surface;
a fluid injection nozzle which is positioned on said flat surface and injects fluid toward said observation window;
a first inclined plane which is disposed around at least a part of the periphery of said observation window facing said fluid injection nozzle and inclines such that whose height gradually decreases from the periphery of said observation window toward outside; and
a back-flow prevention member which is provided on the counter side from said fluid injection nozzle across said observation window so as to prevent that fluid injected from said fluid injection nozzle and passed the surface of said observation window flows backwardly from said flat surface toward the surface of said observation window.

2. An endoscope claimed in claim 1, wherein said back-flow prevention member is a plane which is positioned at the periphery of said observation window and stands from said flat surface in a height direction in which the surface of said observation window is projected, said plane being a vertical plane standing vertically from said flat surface or being a second inclined plane having an acute angle with said flat surface.

3. An endoscope claimed in claim 1, wherein said first inclined plane has a width covering an injection area of fluid from said fluid injection nozzle.

4. An endoscope claimed in claim 1, wherein said back-flow prevention member is disposed along the periphery of said observation window.

5. An endoscope claimed in claim 4, wherein said back-flow prevention member is disposed along a rest portion of the periphery of said observation window where said first inclined plane is not disposed.

6. An endoscope claimed in claim 5, wherein a link surface which smoothly links between said first inclined plane and said second inclined plane is formed at a boundary between said first inclined plane and said back-flow prevention member, so that there is no edge portion at the boundary.

7. An endoscope claimed in claim 5, wherein each of said first inclined plane and said back-flow prevention member covers approximately each half of the periphery of said observation window.

8. An endoscope claimed in claim 5, wherein said back-flow prevention member covers between said observation window and an illumination window disposed near said observation window.

9. An endoscope claimed in claim 5, wherein the back-flow prevention member side of said flat surface with reference to the boundary between said back-flow prevention member and said first inclined plane has a height lower than that of the first inclined plane side of said flat surface.

10. An endoscope claimed in claim 1, wherein said back-flow prevention member is integrally formed with a member on which said flat surface is formed.

11. An endoscope claimed in claim 1, wherein said back- flow prevention member is formed on another member which is different from a member on which said flat surface is formed.

12. An endoscope claimed in claim 11, wherein said other member is a lens barrel to which said observation window is attached, the peripheral surface of said lens barrel working as said back-flow prevention member.

13. An endoscope claimed in claim 11, wherein said other member is said observation window itself, the flange of said observation window working as said back-flow prevention member.

14. An endoscope claimed in claim 1, wherein said inserter comprises:
a suction opening which is provided on said flat surface and sucks liquid around it; and
a third inclined plane which is provided around said observation window and inclines toward said suction opening, with passing through between a projection projected from said flat surface and said observation window, and around said back-flow prevention member.

15. An endoscope claimed in claim 1, wherein said inserter comprises a connecting member which is provided around said observation window and connects between a projection projected from said flat surface and said observation window.

16. An endoscope claimed in claim 15, wherein said connecting member covers an area between said observation window and said projection, and wherein said back-flow prevention member is a cut-out formed by cutting a part of said connecting member positioned at the counter side from said fluid injection nozzle across said observation window.

17. An endoscope claimed in claim 16, wherein said flat surface is formed such that the cut-out side thereof has a height lower than that of the first inclined plane side thereof.
